(19) United States
(12) Patent Application Publication
(10) Pub. No.: US 2009/0149569 A1
(43) Pub. Date: Jun. 11, 2009

(54) SURFACE ENGINEERING OF TISSUE GRAFT MATERIALS FOR ENHANCED POROSITY AND CELL ADHESION

(76) Inventors: V. Prasad Shastri, Nashville, TN (US); Henrique Franca Diniz Oliveira, Belo Horizonte (BR)

Correspondence Address: Ballard Spahr Andrews & Ingersoll, LLP, SUITE 1000, 999 PEACHTREE STREET, ATLANTA, GA, 30309-3915, US

(21) Appl. No.: 12/176,765

(22) Filed: Jul. 21, 2008

Related U.S. Application Data
(60) Provisional application No. 60/950,873, filed on Jul. 19, 2007.

(51) Int. Cl. A61F 2/02

(52) U.S. Cl. 523/113

(57) ABSTRACT

In one aspect, the invention relates to providing enhanced application tissue graft materials in regenerative medicine through improved cellular interactions. Biocompatible implant materials, methods for preparing biocompatible implant materials, methods for using same, and methods for treating tissue injury are disclosed. This abstract is intended as a scanning tool for purposes of searching in the particular art and is not intended to be limiting of the present invention.

### CROSS-REFERENCE TO RELATED APPLICATIONS

This application claims the benefit of U.S. Application No. 60/950,873, filed Jul. 19, 2007, which is hereby incorporated herein by reference in its entirety.

### BACKGROUND

In the healing arts, there is often a need for an implant or graft material to replace, repair, or reconstruct tissues, in particular, hard tissues such as bone. For example, hard-tissue implant materials have been used in medicine and veterinary medicine as prosthetic bone materials to repair injured or diseased bone. Hard tissue implant materials are also used in the construction of prosthetic joints to fix the prosthetic joints to bones. In the dental art, hard tissue implant materials are used in the reconstruction of jaw bone damages caused by trauma, disease, or tooth loss; in the replacement or augmentation of the edentulous ridge; in the prevention of jaw bone loss by socket grafting; and in the treatment of periodontal bone void defects. In orthopedics, hard tissue implant materials are used in the reconstruction of bone structure caused by trauma, disease, or surgery. For surgical procedures such as intervertebral diskectomy, the intervertebral disk is removed to provide access in removing the offending tissue, or bone osteophytes. In a spinal fusion procedure, it may be required to fix the vertebrae together to prevent movement and maintain a space originally occupied by the intervertebral disk.

Graft materials, such as bone to be use for spinal fusion following a diskectomy, can be removed from another portion of the patient's body, termed an autograft. The use of bone taken from the patient's body has the important advantage of avoiding rejection of the implant, but has several shortcomings. There is always a risk in opening a second surgical site in obtaining the implant, which can lead to infection or pain for the patient, and the site of the implant is weakened by the removal of bony material. The bone implant may not be perfectly shaped and placed, leading to slippage or absorption of the implant, or failure of the implant to fuse with the vertebrae.

Other options for a graft source of the implant are bone removed from cadavers, termed allograft, or from other species, termed a xenograft. In these cases while there is the benefit of not having a second surgical site as a possible source of infection or pain, there is increased difficulty of the graft rejection and the risk of transmitting communicable diseases.

An alternative approach is the use of a synthetic graft material that is biologically compatible with the body and the target tissue. For example, over the last decade, polymeric materials have been used widely as bone graft materials. These materials are bio-inert, biocompatible, can serve as a temporary scaffold to be replaced by host tissue over time, and can be degraded by hydrolysis or by other means to non-toxic products. However, needed in the art are compositions and methods for modifying the surface of graft materials improve porosity and cell attachment.

### SUMMARY

In accordance with the purpose(s) of the invention, as embodied and broadly described herein, the invention, in one aspect, relates to providing enhanced application tissue graft materials (e.g., Bioplant-HTR) in regenerative medicine through improved cellular interactions.

Disclosed are biocompatible implant materials comprising a polymeric tissue graft material having a surface and a plurality of nonpolymeric porous particles disposed at the surface.

A biocompatible implant material comprising a hollow microsphere having a surface and an inner cavity, the microsphere comprising an outer layer consisting essentially of poly(hydroxyethyl methacrylate) and an inner layer consisting essentially of poly(methyl methacrylate); and a plurality of coral particles with a particle size of from about 180 μm to about 250 μm disposed at the surface, wherein the implant material has a diameter of from about 500 μm to about 1000 μm.

Also disclosed are methods for preparing a biocompatible implant material, the method comprising the steps of contacting a polymeric tissue graft material with an organic solvent and mixing the microsphere with a plurality of nonpolymeric porous particles.

Also disclosed are methods for preparing a biocompatible implant material, the method comprising the steps of providing a microsphere comprising an outer layer consisting essentially of poly(hydroxyethyl methacrylate) and an inner layer consisting essentially of poly(methyl methacrylate); roughening the surface of the microsphere by contacting the microsphere with a dichloromethane/ethanol (30%/70% v/v) solution, thereby providing a roughened microsphere; and forming a composite microsphere by: exposing the roughened uncoated microsphere to a dichloromethane/ethanol (70%/30% v/v) solution and mixing the roughened uncoated microsphere with coral particles with a particle size of from about 180 μm to about 250 μm.

Also disclosed are methods for preparing a biocompatible implant material, the method comprising the steps of providing a microsphere comprising an outer layer consisting essentially of poly(hydroxyethyl methacrylate), an inner layer consisting essentially of poly(methyl methacrylate), and a calcium hydroxide coating; removing the calcium hydroxide coating by contacting the microsphere with an acidic solution for a period of time sufficient to substantially remove the coating, thereby providing a substantially uncoated microsphere; roughening the surface of the microsphere by contacting the microsphere with a dichloromethane/ethanol (30%/70% v/v) solution, thereby providing a roughened uncoated microsphere; and forming a composite microsphere by: exposing the roughened uncoated microsphere to a dichloromethane/ethanol (70%/30% v/v) solution and mixing the roughened uncoated microsphere with coral particles with a particle size of from about 180 μm to about 250 μm.

Also disclosed are the products of the disclosed methods.

Also disclosed are methods comprising providing a polymeric tissue graft material having a surface wherein a plurality of nonpolymeric porous particles are disposed at said surface, and mixing cells with the polymeric tissue graft material the under conditions suitable to promote cell growth

Also disclosed are methods comprising administering cells to a polymeric tissue graft material having a surface wherein a plurality of nonpolymeric porous particles are disposed at said surface under conditions suitable to promote cell growth.

Also disclosed are kits comprising the disclosed materials.

Also disclosed are pharmaceutical compositions comprising the disclosed materials.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### BRIEF DESCRIPTION OF THE FIGURES

The accompanying figures, which are incorporated in and constitute a part of this specification, illustrate several aspects and together with the description serve to explain the principles of the invention.

FIG. 1 shows a schematic of exemplary modification steps for a tissue graft materials (e.g., HTR).

FIG. 2 shows a schematic of exemplary steps in coating a cell plate and imaging the cells.

FIG. 3 shows a SEM image (100× magnification) and zoomed image (1000× magnification) of the surface of the unmodified HTR particle. Unmodified HTR particle exhibits a rough surface.

FIG. 4 shows a SEM image (100× and 1000× magnification) of the surface of the HTR particle with the coating removed. Removal of the Ca(OH)₂ layer by acid-washing yielded a smoother and less homogeneous PMMA surface.

FIG. 5 shows a SEM image (100× and 1000× magnification) of the surface of the PMMA put in dichloromethane solution. Dichloromethane partially dissolved the PMMA, creating roughness and ridges on the particle surface.

FIG. 6 shows a SEM image (100× and 1000× magnification) of the surface of PMMA particles attached with coral. The addition of coral created additional roughness on the particle surface.

FIG. 7 shows tissue graft material (here, HTR) particles and cells imaged with fluorescence microscope (5×). HTR=13.9±7 cells/field of vision. 46.6% of the visible cells had attached to the particle surface.

FIG. 8 shows PMMA particles and cells imaged with fluorescence microscope (5×). PMMA=6.75±3.7 cells/field of vision. 23.4% of the visible cells had attached to the particle surface.

FIG. 9 shows PMMA+dichloromethane particles and cells imaged with fluorescence microscope (5×). PMMA+DCM=13.9±10 cells/field of vision. 69.1% of the visible cells had attached to the particle surface.

FIG. 10 shows PMMA+coral particles and cells imaged with fluorescence microscope (5×). PMMA+coral=8.1±3 cells/field of vision. 70.2% of the visible cells had attached to the particle surface.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DESCRIPTION

The present invention can be understood more readily by reference to the following detailed description of the invention and the Examples included therein.

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which may need to be independently confirmed.

### A. DEFINITIONS

As used in the specification and the appended claims, the singular forms “a,” “an” and “the” include plural referents unless the context clearly dictates otherwise. Thus, for example, reference to “a functional group,” “an alkyl,” or “a residue” includes mixtures of two or more such functional groups, alkyls, or residues, and the like.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

A residue of a chemical species, as used in the specification and concluding claims, refers to the moiety that is the resulting product of the chemical species in a particular reaction scheme or subsequent formulation or chemical product, regardless of whether the moiety is actually obtained from the chemical species. Thus, an ethylene glycol residue in a polyester refers to one or more —OCH₂CH₂O— units in the polyester, regardless of whether ethylene glycol was used to prepare the polyester. Similarly, a sebacic acid residue in a polyester refers to one or more —CO(CH₂)₈CO— moieties in the polyester, regardless of whether the residue is obtained by reacting sebacic acid or an ester thereof to obtain the polyester.

The subject of the herein disclosed methods can be a vertebrate, such as a mammal, a fish, a bird, a reptile, or an amphibian. Thus, the subject of the herein disclosed methods can be a human, primate, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. A patient refers to a subject afflicted with a disease or disorder. The term “patient” includes human and veterinary subjects.

As used herein, the terms “optional” or “optionally” means that the subsequently described event or circumstance can or can not occur, and that the description includes instances where said event or circumstance occurs and instances where it does not.

As used herein, the term “substantially” means that the subsequently described event or circumstance completely occurs or that the subsequently described event or circumstance generally, typically, or approximately occurs. For example, when the specification discloses that method steps are performed substantially simultaneously, a person skilled in the relevant art would readily understand that the steps need not be synchronized. Rather, this term conveys to a person skilled in the relevant art that the method steps can be synchronized, can be overlapping in time, or can be separated by a technically insignificant (e.g., commercially insignificant) amount of time.

By “treatment” is meant the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed. In certain aspects, this term can be synonymous with the language “preventative treatment.”

As used herein, the term “alleviate” or “alleviating” refers to lightening or lessening the severity of a symptom, condition, or disorder. For example, a treatment that reduces the severity of pain in a subject can be said to alleviate pain. It is understood that, in certain circumstances, a treatment can alleviate a symptom or condition without treating the underlying disorder. In certain aspects, this term can be synonymous with the language “palliative treatment.”

As used herein, the term “diagnosed with” a condition refers to having been subjected to a physical examination by a person of skill, for example, a medical doctor (e.g., physician or veterinarian), and found to have the condition. It is also specifically contemplated that a subject (e.g., a mammal, a human) can be identified with such condition.

As used herein, the term “diagnosed with a need for” a treatment refers to having been subjected to a physical examination by a person of skill, for example, a physician, and found to have a condition that can be diagnosed or treated by the treatment. It is also specifically contemplated that a subject (e.g., a mammal, a human) can be identified with a need for such treatment.

As used herein, the term “biocompatible” refers to materials, or by-products thereof, that are non-toxic and do not elicit a strong immunological reaction against the material. However, the term “biocompatible” does not necessarily exclude materials that elicit an immunogenic response such that the reaction is not adverse.

As used herein, the term “biodegradable” refers to materials which are enzymatically or chemically degraded, or degraded by dissociative processes such as unlinking of an ionically cross-linked material, or dissociation of physically cross-linked structures in vivo into simpler chemical species or species that can be processed by the body through excretory mechanism's.

As used herein, the terms “implanting” or “implantation” refer to any method of introducing a composition, for example a graft material, into a subject. Such methods are well known to those skilled in the art and include, but are not limited to, surgical implantation or endoscopic implantation. The term can include both sutured and bound implantation.

As used herein, the term “alloplast” refers to one example of a tissue graft material. In one aspect, an alloplast is an inert material. In a further aspect, an alloplast is a degradable material, for example a material that absorbs slowly within a subject. In a yet further aspect, an alloplast is a non-degradable material, for example a material that does not substantially absorbs within a subject.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, the term “effective amount” refers to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side affects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

As used herein, a “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity may be maintained, for example, by the use of coating materials such as lecithin, by the maintenance of the required particle size in the case of dispersions and by the use of surfactants. These compositions may also contain adjuvants such as preservatives, wetting agents, emulsifying agents and dispersing agents. Prevention of the action of microorganisms may be ensured by the inclusion of various antibacterial and antifungal agents such as paraben, chlorobutanol, phenol, sorbic acid and the like. It may also be desirable to include isotonic agents such as sugars, sodium chloride and the like. Prolonged absorption of the injectable pharmaceutical form may be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations may be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds can not be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C—F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

### B. COMPOSITIONS

Provided herein is a biocompatible implant material comprising a polymeric tissue graft material having a surface and a plurality of nonpolymeric porous particles disposed at the surface. The polymeric tissue graft material can comprise a macroporous material. The macroporous material can have pores of greater than about 100 μm. The polymeric tissue graft material can comprise microspheres having diameters of from about 1 μm to about 10,000 μm, from about 50 μm to about 5000 μm, from about 100 μm to about 1000 μm, or from about 50 μm to about 1000 μm. Thus, the microspheres can have diameters of from about 500 μm to about 1000 μm. Thus, the diameters of the microspheres range from about 500 μm to about 1000 μm.

The polymeric tissue graft material can comprise poly(hydroxyethyl methacrylate) and/or poly(methyl methacrylate). The polymeric tissue graft material can comprise a biodegradable polymer. The polymeric tissue graft material can comprise a nonbiodegradable polymer.

The nonpolymeric porous particles can comprise a microporous material. The microporous material can have pores of less than about 100 μm. The microporous material can have pores of from about 0.5 μm to about 20 μm. The nonpolymeric porous particles can have an average particle size of from about 50 μm to about 500 μm, from about 100 μm to about 400 μm, from about 200 μm to about 300 μm, or from about 180 μm to about 250 μm. The nonpolymeric porous particles can have an average particle size of from about 180 μm to about 250 μm. Thus, the particle sizes of the nonpolymeric porous particles range from about 180 μm to about 250 μm.

The nonpolymeric porous particles can comprise a nonmetallic inorganic material. The nonpolymeric porous particles can comprise a ceramic. The nonpolymeric porous particles can comprise coral, shell, pearl, or glass. Thus, the nonpolymeric porous particles can comprise coral.

Thus, provided is a polymeric tissue graft material comprising a microsphere comprising a poly(hydroxyethyl methacrylate) outer layer and a poly(methyl methacrylate) inner layer and wherein the nonpolymeric porous particles comprise coral particles.

Also provided is a biocompatible implant material comprising a polymeric microsphere having a surface and a plurality of ceramic particles disposed at the surface, wherein the implant material has a diameter of from about 500 μm to about 1000 μm.

Also provided is a biocompatible implant material comprising a hollow microsphere having a surface and an inner cavity, the microsphere comprising an outer layer consisting essentially of poly(hydroxyethyl methacrylate) and an inner layer consisting essentially of poly(methyl methacrylate); and a plurality of coral particles with a particle size of from about 180 μm to about 250 μm disposed at the surface, wherein the implant material has a diameter of from about 500 μm to about 1000 μm.

1. Polymeric Tissue Graft Material

The polymeric tissue graft material of the provided implantable composition can be any biocompatible material suitable for implantation into a subject. For example, the polymeric tissue graft material can be a polymeric particle, ceramic, metal, orthopaedic or dental implant, endovascular device, stent, balloon catheter, barrier membrane, surgical mesh, wound dressing, or tissue engineering scaffold. In one aspect, the polymeric tissue graft material is a bone graft material.

There are three main types of bone graft materials: autogenous bone that is naturally osteogenic, osteoinductive, and osteoconductive; allografts (cortical or trabecular), that can be osteoinductive and osteoconductive; and alloplasts (synthetic or natural), which are generally osteoconductive only. The polymeric tissue graft material can be an osteogenic, osteoinductive, or osteoconductive material. Thus, in another aspect of the provided implantable composition, the polymeric tissue graft material is a natural or synthetic alloplast bone grafting material.

There are numerous examples of natural and synthetic alloplast bone grafting materials known in the art, some of which are specifically disclosed herein. Any of these known or newly discovered materials are contemplated for use in the herein disclosed compositions and methods.

For example, the polymeric tissue graft material can comprise a porous matrix of biologically-compatible polymeric particles. In one aspect, calcium hydroxide is distributed in the pores of the matrix. Examples of polymeric particles for use in implants are known and disclosed herein.

U.S. Pat. Nos. 4,535,485 and 4,536,158 are incorporated by reference for the teaching of polymer-based implantable porous prostheses for use as bone or other hard tissue replacement which are composed generally of polymeric particles.

U.S. Pat. No. 4,728,570 is incorporated by reference for the teaching of porous implant material which induces the growth of hard tissue. Based on the '570 patent, Bioplant Inc. (South Norwalk, Conn.) manufactures a very slowly absorbable product called Bioplant® HTR®.

Medical devices made with degradable polyesters such poly(L-lactic acid), poly(glycolic acid), and poly(lactic-co-glycolic acid) are approved for human use by the Food and Drug Administration, and have been used in many medical applications, for example, in sutures.

U.S. Pat. No. 6,511,510 is incorporated herein by reference for the teaching of implantable ceramic materials. Suitable examples of ceramic materials include coral, calcium phosphates, glass ceramics and materials containing calcium phosphates and/or glass ceramics. Examples of calcium phosphates are octacalcium phosphate, apatites, such as hydroxyapatite and carbonate apatite, whitlockites, such as α-tricalcium phosphate and 3-tricalcium phosphate, and combinations thereof.

The polymeric tissue graft material can comprise both macropores and micropores. The total porosity can range from about 0.1% to about 99.99%, including about 20% to about 90%, including from about 40% to about 70%. The macropores of the polymeric tissue graft material can have a size of from about 0.1 mm to 1.5 mm, including from about 0.2 mm and 1 mm. The micropores of the polymeric tissue graft material can have a size of from about 0.05 μm to about 20 μm, including from about 0.5 μm to about 10 μm. Preferably, the micropores are at least located in the macropores. In accordance with this embodiment, the formation of bone tissue is highly promoted. The micropores can be at least present in the surface of the macropores. The microporosity of the material's surface can lie between about 40% and about 60%.

The polymeric tissue graft material can be any graft material known to one skilled in the art, such as a polymeric one. It can be organic or synthetic or a combination thereof. Organic graft materials include autograft, allograft, xenograft or combinations thereof. Cadaver-derived materials are non-limiting examples of allografts. Bovine-derived materials (e.g., Osterograf® N-300 and Osterograf® N-700) are non-limiting examples of xenografts. Synthetic substitutes are also known as alloplasts.

The polymeric alloplast can be a plurality of micron-sized particles (for example having a diameter from about 250 to 900 microns), each particle comprising a core layer comprised of a first polymeric material and a coating generally surrounding the core layer. The coating can comprise a second polymeric material which is hydrophilic and has a composition different from the composition of the first polymeric material. Both polymeric materials in the polymeric alloplast can be biocompatible. The first polymeric material can be an acrylic polymer, such as poly(methyl methacrylate) (PMMA). The PMMA can further include a plasticizer, if desired. The second polymeric material can be a polymeric hydroxyethyl methacrylate (PHEMA). Examples of polymeric particles are disclosed in the '485 patent, the specification of which is hereby incorporated by reference in its entirety.

The bone substitute can be a plurality of calcium hydroxide-treated polymeric micron-sized particles. The quantity of calcium hydroxide is effective to induce the growth of hard tissue in the pores and on the surface of the polymeric micron-sized particles when packed in a body cavity. The calcium hydroxide can form a coating on both the outer and inner surfaces of the polymeric particles.

Examples of procedures for producing the disclosed polymeric tissue graft material are set forth in the specification of the '158 patent. Calcium hydroxide can be introduced into the pores of the micron-sized particles by soaking the particles in an aqueous solution of calcium hydroxide, then removing any excess solution from the particles and allowing the particles to dry.

For example, the polymeric tissue graft material can be Bioplant® HTR,® available from Bioplant Inc. (Norwalk, Conn.), set forth in the '570 patent, which is hereby incorporated by reference in its entirety. The Bioplant® HTR® are microporous particles of calcified (Ca(OH)₂/calcium-carbonate) copolymer of PMMA and PHEMA, with the outer calcium layer interfacing with bone forming calcium carbonate-apatite. The outer diameter of the particles is about 750 μm; the inner diameter is about 600 μm and the pore opening diameter is about 350 μm. Bioplant® HTR® is strong (forces greater than 50,000 lb/in will not crush the Bioplant® HTR® particles), biocompatible and negatively charged (−10 mV) to promote cellular attraction and resist infection. The polymeric tissue graft material can be a form of Bioplant® HTR® comprising particles of calcified (Ca(OH)₂/calcium-carbonate) copolymer of PMMA and PHEMA, with the outer calcium layer interfacing with bone forming calcium carbonate-apatite. In some examples, the Calcium hydroxide layer is removed as exemplified herein.

Thus, the polymeric tissue graft material can comprise an inner layer comprised of a first biologically-compatible polymeric material and an outer layer comprised of a second biologically-compatible polymeric material which generally surrounds the inner layer. The second polymeric material can be hydrophilic and have a composition which is different from the composition of the first polymeric material.

The first polymeric material can be an acrylic polymer. Thus, the first polymeric material can be polymethylmethacrylate (PMMA). The second polymeric material can be a polymeric hydroxyethylmethacrylate (PHEMA). The polymeric hydroxyethylmethacrylate can comprise a copolymer of monomeric hydroxyethylmethacrylate and a cross-linking agent. Cross-linking agents include triethyleneglycol dimethacrylate, tetraethyleneglycol dimethacrylate, diethyleneglycol dimethacrylate, and monoethyleneglycol dimethacrylate. The cross-linking agents can comprise from about 0.1 percent to about 5 percent by weight of the monomeric hydroxyethylmethacrylate.

2. Non-Polymeric Porous Particles

a. Ceramics/Coralline Hydroxyapatite

U.S. Pat. No. 3,299,971 discloses a method of producing a porous synthetic material for use in hard tissue replacement. In this method, a porous carbonate skeletal material of marine life (coral) is converted into a porous hydroxyapatite material through a hydrothermal chemical exchange with a phosphate. The final microstructure of the converted hydroxyapatite material is essentially the same as that of the coral from which it was formed. Consequently, pore size is dependent on the type of coral used. While these porous structures possess the appropriate pore size and pore connectivity for hard tissue in-growth, the structure is limited to that of the selected coral and so the production of implants with a solid shell surrounding the porous network (typical of cortical or long bone, for example) is unobtainable. In addition, the bone grafts manufactured using this technique are characterized by poor mechanical properties and are difficult to handle and shape and cannot be secured using standard fixation techniques.

Interpore® 200 and ProOsteon® Implant 500, also referred to as Replamineform hydroxyapatite and coralline hydroxyapatite, have been found to be useful as bone substitute materials in dental and surgical applications. These materials are essentially non-degradable, yet biocompatible, and resemble the microstructure of animal and human bone. The porosity of these coral derived materials has been characterized as polymodal by means of scanning electron microscope and mercury porosimetery. The macroporosity is characterized by macropores of 100-1000 μm. The microporosity is characterized by spaces between crystallites on the order of 0.1 μm and larger micropores on the order of 1 μm. More information concerning these materials can be found in the article by Drs. Eugene W. White and Edwin C. Shors entitled “Biomaterial Aspects of Interpore-200® Porous Hydroxyapatite,” which appeared in Dental Clinics of North America, Vol. 30, January 1986, pp. 49-67, incorporated herein by reference. While calcium phosphates such as Interpore 200, and ProOsteon® Implant 500 are desirable for many applications, and promote the ingrowth of bone and other tissue into and around the implant, they do not satisfy all of the needs of surgeons using them as bone replacements or implants. U.S. Pat. No. 4,976,736 (White and Shors) (incorporated by reference) also discloses biomaterials useful for orthopedic and dental applications in which two rates of degradation are sought. To accomplish this, the inventors disclose a biomaterial (and method for making such a biomaterial) which has a base portion of calcium carbonate and a surface layer of calcium phosphate or hydroxyapatite. The biomaterial may be machined into various shapes and sizes for orthopedic and dental applications. The biomaterial presents an interface of hydroxyapatite to tissue and body fluids at the site of the surgical defect. The unreacted carbonate behind the interface gradually gets replaced by new bone ingrowth, thereby more completely filling the implant site with the body's own bone material. In one embodiment mentioned in that patent, the macroporosity of the composite is filed with synthetic polymer such as polysulfone, polythylene, silicone rubber or polyurethane, either with positive injection pressure or by vacuum impregnation. After solification of the polymer, the carbonate may optionally be dissolved away with 10acetic acid, leaving behind the polymer that filled the pores.

Porous ceramics can also be manufactured using a variety of other methods. These ceramics, also made from calcium phosphates, can be used as bone graft substitutes. However, they also have mechanical limitations due to the porosity and to the brittle nature of ceramics. Some of these ceramics have microporosity in addition to macroporosity. Examples include U.S. Pat. Nos. 5,348,788; 5,455,100; and 5,487,933.

Tencer et al., in an article entitled, “Bone Ingrowth Into Polymer Coated Porous Synthetic Coralline Hydroxyapatite,” J. Orth. Res. pp. 275-82 (1987), discusses dip-coating the macroporosity or large pores of a coralline hydroxyapatite sample with a polylactic acid (DL-PLA) dilactic-polylactic acid polymer by dipping blocks for 5 seconds in a high (3:1), medium (10:1), or low (30:1) viscosity solution of DL-PLA in chloroform.

The biocompatibility of hydroxyapatite is well established and it is available in dense and porous forms. Coralline hydroxyapatite is widely used as a bone substitute material in oral, periodontal and craniofacial surgery, and has recently been approved for various orthopedic applications, such as bone replacements due to trauma. Other applications are under consideration or investigation. Porous hydroxyapatite promotes bone ingrowth in and around the implant.

U.S. Pat. No. 6,376,573 discloses a method of converting coral-derived calcium carbonate into hydroxyapatite by hydrothermal chemical exchange with a phosphate donor. The resulting synthetic phosphate (hydroxyapatite) converted skeletal material possesses substantially the same macroporosity (˜100-1000, μm pore diameter) of the original carbonate skeletal material from which it was derived, and preserves intact the interconnecting porosity which provides channels and interstices for bone and tissue ingrowth.

The porous ceramic disclosed herein, such as those made from porous carbonate (aragonite) skeletal material of marine life that are comprised predominantly of hydroxyapatite Ca₁₀(PO₄)₆(OH)₂ with some carbonate present, can approximate the carbonate composition of the inorganic component of hard human bone tissue. This hydroxyapatite surface can have osteophilic and osteoconductive properties, and help promote the growth of bone tissue into the porosity or voids in the biomaterial.

The porous ceramic disclosed herein can have a microstructure that is macroporous, completely interconnected, approximating the same pore size as cancellous human bone which would allow permeation of body fluids and blood cells thereinto. Preferably the material includes at least some macropores communicating with the exterior surface of the implant, that is, pores of sufficient size to allow infiltration of blood vessels and other tissues and nutrients necessary to form calcified bone tissue therein. The material can also include micropores, which are pores too small in diameter to permit ingrowth of calcified bone tissue.

As indicated, various porous carbonate skeletal materials, particularly porous carbonate marine skeletal material, may be used herein. Particularly useful, because of the vast quantities available, is the carbonate skeletal material of scleractinian coral Porites. This skeletal material is composed of the calcium carbonate (aragonite), and the average pore size is approximately 200 microns. Other corals of the genera Goniopora, Alveopora, Acropora and others may be suitably employed herein as the source of the calcium carbonate skeletal material for conversion by hydrothermal chemical exchange with a phosphate into hydroxyapatite. Goniopora has an average pore size of about 500 microns, and includes macropores ranging in size from 5 microns to about 1000 microns, making it suitable for orthopedic uses where larger amounts of bone and tissue ingrowth might be beneficial.

Where the carbonate skeletal material is made up of a calcite carbonate marine skeletal material, and where the calcite contains a substantial amount of magnesium associated therewith the hydrothermal chemical exchange produces whitlockite with a phosphate on the surface of the biomaterial. Both materials, however, hydroxyapatite and whitlockite, are useful materials, with the hydroxyapatite being preferred for the manufacture of human implants, such bone fillers and replacements and the like.

A non-limiting list of porous ceramics that can be used herein include: Pro Osteon® (Interpore Cross International, Inc., Irvine, Calif.) comprising monolithic ceramic granules, which are made using coralline calcium carbonate fully or partially converted to HA by a hydrothermal reaction, see D. M. Roy and S. K. Linnehan, Nature, 247, 220-222 (1974); R. Holmes, V. Mooney, R. Bucholz and A. Tencer, Clin. Orthop. Rel. Res., 188, 252-262 (1984); and W. R. Walsh, et al., J. Orthop. Res., 21, 4, 655-661 (2003). VITOSS® (Orthovita, Malvern, Pa.) is provided as monolithic ceramic granules. Norian SRS® (Synthes-Stratec, affiliates across Europe and Latin America) and Alpha-BSM® (ETEX Corp., Cambridge, Mass.) are provided as an injectable pastes. ApaPore® and Pore-SI (ApaTech, London, England) comprise monolithic ceramic granules.

3. Optional Components

The disclosed implantable composition can contain the following optional components.

a. Initiators

The disclosed implantable composition can contain free-radical initiators such as photoinitiators, thermally activated initiators, redox initiator systems, ionic initiators or mixture thereof. Any free-radical initiators or combination of initiators can be used. In a first preferred embodiment, one or more photoinitiator(s) is used. In a second preferred embodiment, one or more redox initiator system(s) is used. In a third preferred embodiment, one or more thermal initiator(s) is used. In a fourth preferred embodiment, one or more photoinitiator(s) is used in combination with one or more redox initiator system(s). In a fifth preferred embodiment, one or more thermal initiator(s) is used in combination with one or more redox initiator system(s). In a sixth embodiment, one or more photoinitiator(s) is used in combination with one or more thermal initiator(s). In a seventh preferred embodiment, one or more photoinitiator(s) and one or more thermal initiator(s) are used in combination with one or more redox initiator system(s).

The concentration of the initiator(s) used depends a number of factors. Non-limiting examples of such factors include the type of the initiator, whether the initiator is used alone or in combination with other initiators, the desirable rate of curing, and how the material is applied. In a preferred embodiment, the concentration of the initiator is between about 0.05% (w/w) to about 5% (w/w) of the crosslinkable prepolymer. For photoinitiator(s) or redox initiator system(s), the concentration of the initiator(s) is preferably less than 1% (w/w) of the crosslinkable prepolymer; more preferably between 0.05 and 0.1% (w/w). For thermal initiator(s), the preferred range is about 1% (w/w) to about 2% (w/w) of the crosslinkable prepolymer.

A photoinitiator is an initiator activated by radiation. Such radiation could be ultraviolet light (e.g., long wavelength ultraviolet light), light in the visible region, focused laser light, infra-red and near-infra-red light, X-ray radiation or gamma radiation. The preferably radiation is light in the visible region and/or near-infra-red region.

Non-limiting examples of the photoinitiators include biocompatible photoinitiators such as beta carotene, riboflavin, Irgacure 651® (2,2-dimethoxy-2-phenylacetophenone), phenylglycine, dyes such as eosin dye, and initiators such as 2,2-dimethyl-2-phenylacetophenone, 2-methoxy-2-phenylacetophenone, and camphorquinone.

Exposure of dyes and co-catalysts such as amines to light generates active species. Light absorption by the dye causes the dye to assume a triplet state; the triplet state subsequently reacts with the amine to form an active species which initiates polymerization. Numerous dyes can be used for photopolymerization. Suitable dyes are well known to those of skill in the art. Preferred dyes include erythrosin, phloxime, rose bengal, thonine, camphorquinone, ethyl eosin, eosin, methylene blue, riboflavin, 2,2-dimethyl-2-phenylacetophenone, 2-methoxy-2-phenylacetophenone, 2,2-dimethoxy-2-phenyl acetophenone, other acetophenone derivatives, and camphorquinone. Suitable co-catalysts include amines such as N-methyldiethanolamine, N,N-dimethylbenzylamine, triethanolamine, triethylamine, dibenzyl amine, N-benzylethanolaamine, N-isopropylbenzylamine. Triethanolamine is a preferred co-catalyst.

A redox initiator system includes an oxidizing agent (also called an oxidizing component) (such as a peroxide) and a reducing agent (also called a reducing component) (such as an aromatic or aliphatic amine). Combining the redox couple results in the generation of an initiating species (such as free radicals or cations) capable of causing curing. Preferably, the redox couples of this invention are activated at temperatures below about 40.degree. C., for example, at room temperature or at the physiological temperature of about 37.degree. C. Generally, the redox couple is partitioned into separate reactive compositions prior to use and then subsequently mixed at the time of use to generate the desired initiating species. Selection of the redox couple is governed by several criteria. For example, a desirable oxidizing agent is one that is sufficiently oxidizing in nature to oxidize the reducing agent, but not excessively oxidizing that it may prematurely react with other components with which it may be combined during storage. Similarly, a desirable reducing agent is one that is sufficiently reducing in nature to readily react with the preferred oxidizing agent, but not excessively reducing in nature such that it may reduce other components with which it may be combined during storage. Oxidation or reduction of the resin with an inappropriate reducing agent or oxidizing agent, respectively, could result in an unstable system that would prematurely polymerize and subsequently provide a limited shelf life. Thus, suitable redox couples individually provide good shelf-life (for example, at least 2 months, preferably at least 4 months, and more preferably at least 6 months in an environment of 5-20.degree. C.), and then, when combined together, generate the desired initiating species for curing or partially curing the curable admixture.

Suitable oxidizing agents include peroxide compounds (I.e., peroxy compounds), including hydrogen peroxide as well as inorganic and organic peroxide compounds (e.g., “per” compounds or salts with peroxoanions). Examples of suitable oxidizing agents include, but are not limited to: peroxides such as benzoyl peroxide, phthaloyl peroxide, substituted benzoyl peroxides, acetyl peroxide, caproyl peroxide, lauroyl peroxide, cinnamoyl peroxide, acetyl benzoyl peroxide, methyl ethyl ketone peroxide, sodium peroxide, hydrogen peroxide, di-tert butyl peroxide, tetraline peroxide, urea peroxide, and cumene peroxide; hydroperoxides such as p-methane hydroperoxide, di-isopropyl-benzene hydroperoxide, tert-butyl hydroperoxide, methyl ethyl ketone hydroperoxide, and 1-hydroxy cyclohexyl hydroperoxide-1, ammonium persulfate, sodium perborate, sodium perchlorate, potassium persulfate, etc.; ozone, ozonides, etc. These oxidizing agents may be used alone or in admixture with one another. Benzoyl peroxide is the preferred oxidizing agent. One or more oxidizing agents may be present in an amount sufficient to provide initiation of the curing process. Preferably, this includes about 0.01 weight percent (wt-%) to about 4.0 wt-%, and more preferably about 0.05 wt-% to about 1.0 wt-%, based on the total weight of all components of the dental material.

A reducing agent has one or more functional groups for activation of the oxidizing agent. Preferably, such functional group(s) is selected from amines, mercaptans, or mixtures thereof. If more than one functional group is present, they may be part of the same compound or provided by different compounds. A preferred reducing agent is a tertiary aromatic amine (e.g., N,N-dimethyl-p-toluidine (DMPT) or N,N-bis(2-hydroxyethyl)-p-toluidine (DHEPT)). Examples of such tertiary amines are well known in the art and can be found, for example, at WO 97/35916 and U.S. Pat. No. 6,624,211. Another preferred reducing agent is a mercaptan, which can include aromatic and/or aliphatic groups, and optionally polymerizable groups. Preferred mercaptans have a molecular weight greater than about 200 as these mercaptans have less intense odor. Other reducing agents, such as sulfinic acids, formic acid, ascorbic acid, hydrazines, and salts thereof, can also be used herein to initiate free radical polymerization.

If two or more reducing agents are used, they are preferably chosen such that at least one has a faster rate of activation than the other(s). That is, one causes a faster rate of initiation of the curing of the curable admixture than the other(s).

Electrochemical oxidation potentials of reducing agents and reduction potentials of oxidizing agents are useful tools for predicting the effectiveness of a suitable redox couple. For example, the reduction potential of the oxidant (i.e., oxidizing agent) benzoyl peroxide is approximately −0.16 volts vs. a saturated calomel electrode (SCE). Similarly, the oxidation potential (vs. SCE) for a series of amines has been previously established as follows: dihydroxyethyl-p-toluidine ((DHEPT), 0.76 volt), 4-t-butyl dimethylaniline ((t-BDMA), 0.77 volt), 4-dimethylaminophenethanol ((DMAPE), 0.78 volt), triethylamine ((TEA, 0.88 volt), 3-dimethylaminobenzoic acid ((3-DMAB) 0.93 volt), 4-dimethylaminobenzoic acid ((4-DMAB, 1.07 volts), ethyl p-dimethylaminobenzoate ((EDMAB), 1.07 volts), 2-ethylhexyl p-dimethylaminobenzoate ((EHDMAB), 1.09 volts) and 4-dimethylaminobenzoate ((DMABA), 1.15 volts). The ease of oxidation (and subsequent reactivity) increases as the magnitude of the oxidation decreases. Suitable amine reducing agents in combination with benzoyl peroxide generally include aromatic amines with reduction potentials less than about 1.00 volt vs. SCE. Less effective oxidants than benzoyl peroxide such as lauroyl peroxide (reduction potential=−0.60 volt) are poorer oxidizing agents and subsequently react more slowly with aromatic amine reducing agents. Suitable aromatic amines for lauroyl peroxide will generally include those less than about 0.80 volt vs SCE.

Non-limiting examples of an thermal initiator include a peroxydicarbonate, persulfate (e.g., potassium persulfate or ammonium persulfate), an azo initiator such as azosisobutyronitrile (AIBN), and various peroxides (e.g., benzoyl peroxide). Thermally activated initiators, alone or in combination with other type of initiators, are most useful where light can not reach (e.g., deep within the curable admixture).

b. Excipients

One or more excipients can be incorporated into the implantable compositions disclosed herein. Non-limiting examples of such excipients include Ca(OH).sub.2, demineralized bone powder or particles, hydroxyapatite powder or particles, coral powder, resorbable and non-resorbable hydroxyapatite, calcium phosphate particles, .alpha.-tricalcium phosphate, octacalcium phosphate, calcium carbonate, and calcium sulfate. Preferably, such excipients can neutralize the acid generated during the degradation of a biodegradable polymer and maintain a physiological pH value suitable for bone formation. Preferably, such excipient is alkaline in nature so that it can neutralize the acid generated in the biodegradation process and help to maintain a physiological pH value.

c. Porosity Forming Agents

One or more substances that promote pore formation can be incorporated into the implantable compositions disclosed herein. Non-limiting examples of such substances include: particles of inorganic salts such as NaCl, CaCl.sub.2, porous gelatin, carbohydrate (e.g., monosaccharide), oligosaccharide (e.g., lactose), polysaccharide (e.g., a polyglucoside such as dextrane), gelatin derivative containing polymerizable side groups, porous polymeric particles, waxes, such as paraffin, bees wax, and carnuba wax, and wax-like substances, such as low melting or high melting low density polyethylene (LDPE), and petroleum jelly. Other materials include hydrophilic materials such as PEG, alginate, bone wax (fatty acid dimers), fatty acid esters such as mono-, di-, and tri-glycerides, cholesterol and cholesterol esters, and naphthalene. In addition, synthetic or biological polymeric materials such as proteins can be used.

The size or size distribution of the porosity forming agent particles used in the invention can vary according to the specific need. For example, the particle size can be less than about 5000 μm. The particle size can be between about 500 and about 5000 μm. The particle size can be between about 25 and about 500 μm. The particle size can be between about 100 and 250 μm.

d. Therapeutic Agents

One or more preventive or therapeutic active agents and salts or esters thereof formation can be incorporated into the implantable compositions disclosed herein, including but not limited to: 1) antipyretic analgesic anti-inflammatory agents (which are discussed in greater detail, with additional examples, below), including non-steroidal anti-inflammatory drugs (NSAIDs) such as indomethacin, aspirin, diclofenac sodium, ketoprofen, ibuprofen, mefenamic acid, azulene, phenacetin, isopropylantipyrin, acetaminophen, benzydamine hydrochloride, phenylbutazone, flufenamic acid, mefenamic acid, sodium salicylate, choline salicylate, sasapyrine, clofezone or etodolac; and steroidal drugs such as dexamethasone, dexamethasone sodium sulfate, hydrocortisone, prednisolone; 2) antibacterial and antifungal agents such as penicillin, ampicillin, amoxicillin, cefalexin, erythromycin ethylsuccinate, bacampicillin hydrochloride, minocycline hydrochloride, chloramphenicol, tetracycline, erythromycin, fluconazole, itraconazole, ketoconazole, miconazole, terbinafine; nlidixic acid, piromidic acid, pipemidic acid trihydrate, enoxacin, cinoxacin, ofloxacin, norfloxacin, ciprofloxacin hydrochloride, or sulfamethoxazole trimethoprim; 3) anti-viral agents such as trisodium phosphonoformate, didanosine, dideoxycytidine, azido-deoxythymidine, didehydro-deoxythymidine, adefovir dipivoxil, abacavir, amprenavir, delavirdine, efavirenz, indinavir, lamivudine, nelfinavir, nevirapine, ritonavir, saquinavir or stavudine; 4) high potency analgesics such as codeine, dihydrocodeine, hydrocodone, morphine, dilandid, demoral, fentanyl, pentazocine, oxycodone, pentazocine or propoxyphene; and 5) salicylates which can be used to treat heart conditions or as an anti-inflammatory.

The agents can be incorporated in the composition directly, or can be incorporated in microparticles which are then incorporated in the composition. Incorporating the agents in microparticles can be advantageous for those agents that are reactive with one or more of the components of the composition.

e. Diagnostic Agents

One or more diagnostic agents can be incorporated into the implantable compositions disclosed herein. Diagnostic/imaging agents can be used which allow one to monitor bone repair following implantation of the compositions in a patient. Suitable agents include commercially available agents used in positron emission tomography (PET), computer assisted tomography (CAT), single photon emission computerized tomography, X-ray, fluoroscopy, and magnetic resonance imaging (MRI).

Examples of suitable agents useful in MRI include the gadolinium chelates currently available, such as diethylene triamine pentaacetic acid (DTPA) and gadopentotate dimeglumine, as well as iron, magnesium, manganese, copper and chromium.

Examples of suitable agents useful for CAT and X-rays include iodine based materials, such as ionic monomers typified by diatrizoate and iothalamate, non-ionic monomers such as iopamidol, isohexyl, and ioversol, non-ionic dimers, such as iotrol and iodixanol, and ionic dimers, for example, ioxagalte.

These agents can be detected using standard techniques available in the art and commercially available equipment.

f. Bioactive Agent

The herein disclosed implantable composition can further comprise a bioactive agent. The bioactive agent can be any agent such as a molecule, protein, nucleic acid, transfecting agent (vector), therapeutic agent, or diagnostic agent that is suitable for release into tissue by the disclosed implantable composition. Preferred bioactive agents promote tissue growth and/or healing, prevent infection, prevent inflammation, or aid in diagnosis. Other known or newly discovered bioactive agents suitable for release from an implant are considered for use herein.

For example, the bioactive agent can comprise one or more growth factors, cytokines, and/or hormones. The agents can include, for example, proteins originating from various animals including humans, microorganisms and plants, as well as those produced by chemical synthesis and using genetic engineering techniques. As used herein, a “growth factor” includes any soluble factor that regulates or mediates cell proliferation, cell differentiation, tissue regeneration, cell attraction, wound repair and/or any developmental or proliferative process. For example, the bioactive agent can comprise fibroblast growth factor-2 (FGF-2), fibroblast growth factor-1 (FGF-1), epidermal growth factor (EGF), heparin binding growth factor (HBGF), Placental Growth Factor (P1GF), vascular endothelial growth factor (VEGF), transforming growth factor-alpha (TGF-α), transforming growth factor-beta (TGF-β), insulin-like growth factor (IGF-I, IGF-II), platelet derived growth factor (PDGF), leukemia inhibitory factor (LIF), and/or platelet rich plasma (PRP). For example, the bioactive agent can comprise various interferons, including interferon-α, -β, and γ, and/or interleukin-2 and -3. For example, the bioactive agent can comprise insulin, growth hormone-releasing factor, and/or calcitonin.

In one aspect, the agents can promote and/or induce bone formation. Non-limiting examples of suitable bone promoting materials include growth factors such as BMP (Sulzer Orthopedics), BMP-2 (Medtronic/Sofamor Danek), bFGF (Orquest/Anika Therapeutics), Epogen (Amgen), granulocyte colony-stimulating factor (G-CSF) (Amgen), Interleukin growth factor (IGF)-1 (Celtrix Pharmaceuticals), osteogenic protein (OP)-1 (Creative BioMolecules/Stryker Biotec), platelet-derived growth factor (PDGF) (Chiron), stem cell proliferation factor (SCPF) (University of Florida/Advanced Tissue Sciences), recombinant human interleukin (rhIL) (Genetics Institute), transforming growth factor beta (TGF-β) (Collagen Corporation/Zimmer Integra Life Sciences), and TGF-β-3 (OSI Pharmaceuticals). Bone formation may be reduced from several months to several weeks. In orthopedic and dental applications, bone regenerating molecules, seeding cells, and/or tissue can be incorporated into the compositions. For example bone morphogenic proteins such as those described in U.S. Pat. No. 5,011,691, the disclosure of which is incorporated herein by reference, can be used in these applications.

TGF-β superfamily proteins are expressed during bone and joint formation and have been implicated as endogenous regulators of skeletal development. They are also able to induce ectopic bone and cartilage formation and play a role in joint and cartilage development (Storm E E, Kingsley D M. Dev Biol. 1999 May 1; 209(1): 1-27; Shimaoka et al., J Biomed Mater Res A. 200468(1):168-76; Lee et al., J Periodontol. 2003 74(6):865-72). The BMP proteins that may be used include, but are not limited to, BMP-1 or a protein from one of the three subfamilies. BMP-2 (and the recombinant form rhBMP2) and BMP-4 have 80% amino acid sequence homology. BMP-5, -6, and -7 have 78% amino acid sequence homology. BMP-3 is in a subfamily of its own. Normal bone contains approximately 0.002 mg BMP/kg bone. For BMP addition to induce bone growth at an osseous defect, 3 to 3.5 mg BMP has been found to be sufficient, although this number may vary depending upon the size of the defect and the length of time it will take for the BMP to release. Additional carriers for the BMP may be added, and include, for example, inorganic salts such as a calcium phosphate or CaO₄S. (Rengachary, S S, Neurosurg. Focus, 13(6), 2 (2002)). Particular GDFs useful in the present compositions include, but are not limited to GDF-1; GDF-3 (also known asVgr-2); the subgroup of related factors: GDF-5, GDF-6, and GDF-7; GDF-8 and highly related GDF-11; GDF-9 and -9B; GDF-10; and GDF-15 (also known as prostate-derived factor and placental bone morphogenetic protein).

It is important for the bioactive agent to remain active through the polymerization process. For example, many enzymes, cytokines, etc. are sensitive to the radiation used to cure polymers during photopolymerization. The method provided in Baroli et al., J. Pharmaceutical Sci. 92:6 1186-1195 (2003) can be used to protect sensitive molecules from light-induced polymerization. This method provides protection using a gelatin-based wet granulation. This technique may be used to protect the bone promoting agent incorporated into the polymer composition.

In some aspects of the provided implantable composition, the bioactive agent is incorporated into a polymeric composition. Thus, the in one aspect, the bioactive agent can be protected from polymerization-induced damage.

In addition to possible light-induced alterations such as photo-oxidation during photopolymerization, sensitive molecules may be chemically altered upon reacting with monomers, matrix components, and polymerizing species. See Davies M J, Truscott R J W, “Photo-oxidation of proteins and its role in cataractogenesis,” J Photochem Photobio B: Biology 63, 114-125 (2001), herein incorporated by reference. Denaturation reactions are of significance, because entrapped drugs may lose their activity or trigger an immune response. See McNally E J, editor, “Protein formulation and delivery,” New York: Marcell Dekker, Inc. (2000); Cleland J L, Powell M F, Shire S J, “The development of stable protein formulations: A close look at protein aggregation, deamination, and oxidation,” Crit Rev Ther Drug Carrier Syst 10(4), 307-377 (1993); all herein incorporated by reference. Although some studies have shown that proteins can be released from photopolymerized matrices, there are few reports of enzyme entrapment. See Mellot M B, Searchy C, Pishko M V, “Release of protein from highly cross-linked hydrogels of poly(ethylene glycol)diacrylate fabricated by UV polymerization,” Biomaterials 22, 929-941 (2001); Elisseeff J, McIntosh W, Anseth K, Langer R, “Cogelation of hydrolysable cross-linkers and poly(ethylene oxide) dimethacrylate and their use as controlled release vehicles,” in Dinh S M, DeNuzzio J D, Comfort A R, editors, “Intelligent materials for controlled release,” Washington D.C.: ACS, 1-13 (1999); An Y, Hubbell J A, “Intraarterial protein delivery via intimally-adherent bilayer hydrogels,” J Controlled Release 64, 205-215 (2000); Elisseeff J, McIntosh W, Fu K, Blunk T, Langer R, “Controlled-release of IGF-I and TGF-β1 in a photopolymerizing hydrogel for cartilage tissue engineering,” J Orthop Res 19(6), 1098-1104 (2001); all herein incorporated by reference. Nevertheless, in these latter cases, no quantitative assessment was made regarding the extent of enzyme inactivation or enzyme structure modification.

U.S. Pat. No. 5,902,599 is incorporated by reference for the teaching of methods for protecting sensitive therapeutic agents from light-induced polymerization when incorporated in a polymer composition.

As disclosed herein, the bioactive molecules of the implantable composition can be admixed with a photopolymerizable monomer. Thus, in one aspect, the bioactive molecules can be shielded from the monomers by an insoluble material that undergoes a solid-gel transition at body temperature. Thus, the insoluble material can be insoluble in the monomer. Upon polymerization, the monomers produce a cross-linked structure and the shielded bioactive molecules are protected from attack in the polymerizing environment.

The photopolymerizable monomer may belong to any class of compounds, may be of any molecular weight, and may react directly or indirectly to any electromagnetic radiation by polymerizing. In certain embodiments, electromagnetic radiation is comprised under UV, Visible or IR spectrum. When reacting indirectly, a suitable system of one, or a mixture of, photoinitiators and accelerators may be responsible of the radiation energy transfer to the monomer. In certain other embodiments, photoinitiators may include radical polymerization by either photoclevage or hydrogen abstraction, or cationic photopolymerization.

The insoluble material may be a gelatin, collagen, natural polymer or synthetic polymer. The bioactive molecules can be shielded by the insoluble material by granulation, spray drying, spray chilling, lyophilization, coating vapor deposition (CVD), compression, microencapsulation, coating, subcoating, sealing, coacervation, suspension, precipitation, cogelation, gelation, inclusion in pre-formed delivering systems, inclusion into matrix and micromatrix, or evaporation.

Thus, the bioactive agent can be admixed with photo-polymerizable monomers, wherein the bioactive agent is shielded from the monomers by an insoluble material that undergoes a solid-gel transition at body temperature, wherein upon polymerization, the monomers produce a cross-linked structure and the shielded bioactive molecules are protected from attack in the polymerized environment.

The bioactive agent can be released from the composition over a period of hours or days. For example, 50% of the bioactive agent can be release from the composition in at least about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 36 hours, 2 days, 3 days, 4 days, 5 days, 6 days, 7 days, 8 days, 9 days, 10 days, or longer.

g. Therapeutic Agents

The bioactive agent of the provided implantable composition can comprise one or more pharmaceutically active agents. As used herein, the term “pharmaceutically active agent” includes a “drug” and means a molecule, group of molecules, complex or substance administered to an organism for diagnostic, therapeutic, preventative medical, or veterinary purposes. This term includes human and animal pharmaceuticals, treatments, remedies, nutraceuticals, cosmeceuticals, biologicals, devices, diagnostics and contraceptives, including preparations useful in clinical and veterinary screening, prevention, prophylaxis, healing, wellness, detection, imaging, diagnosis, therapy, surgery, monitoring, cosmetics, prosthetics, forensics and the like. This term may also be used in reference to agriceutical, workplace, military, industrial and environmental therapeutics or remedies comprising selected molecules or selected nucleic acid sequences capable of recognizing cellular receptors, membrane receptors, hormone receptors, therapeutic receptors, microbes, viruses or selected targets comprising or capable of contacting plants, animals and/or humans. This term can also specifically include nucleic acids and compounds comprising nucleic acids that produce a bioactive effect, for example deoxyribonucleic acid (DNA), ribonucleic acid (RNA), or mixtures or combinations thereof, including, for example, DNA nanoplexes, antisense molecules, aptamers, ribozymes, triplex forming molecules, RNAi, and external guide sequences. Pharmaceutically active agents include the herein disclosed categories and specific examples. It is not intended that the category be limited by the specific examples. Those of ordinary skill in the art will recognize also numerous other compounds that fall within the categories and that are useful according to the invention.

Thus, the bioactive agent of the provided implantable composition can comprise one or more preventive or therapeutic active agents and salts or esters thereof, including but not limited to: antipyretic analgesic anti-inflammatory agents, including non-steroidal anti-inflammatory drugs (NSAIDs) such as indomethacin, aspirin, diclofenac sodium, ketoprofen, ibuprofen, mefenamic acid, azulene, phenacetin, isopropylantipyrin, acetaminophen, benzydamine hydrochloride, phenylbutazone, flufenamic acid, mefenamic acid, sodium salicylate, choline salicylate, sasapyrine, clofezone or etodolac; and steroidal drugs such as dexamethasone, dexamethasone sodium sulfate, hydrocortisone, or prednisolone; antibacterial and antifungal agents such as penicillin, ampicillin, amoxicillin, cephalexin, erythromycin ethylsuccinate, bacampicillin hydrochloride, minocycline hydrochloride, chloramphericol, tetracycline, erythromycin, fluconazole, itraconazole, ketoconazole, miconazole, terbinafine; nlidixic acid, piromidic acid, pipemidic acid trihydrate, enoxacin, cinoxacin, ofloxacin, norfloxacin, ciprofloxacin hydrochloride, sulfamethoxazole, or trimethoprim; anti-viral agents such as trisodium phosphonoformate, didanosine, dideoxycytidine, azido-deoxythymidine, didehydro-deoxythymidine, adefovir dipivoxil, abacavir, amprenavir, delavirdine, efavirenz, indinavir, lamivudine, nelfinavir, nevirapine, ritonavir, saquinavir or stavudine; high potency analgesics such as codeine, dihydrocodeine, hydrocodone, morphine, dilandid, demoral, fentanyl, pentazocine, oxycodone, pentazocine or propoxyphene; anti-proliferative agent such as taxol; and salicylates which can be used to treat heart conditions or as an anti-inflammatory.

The agents can be incorporated in the disclosed composition directly, or can be incorporated in microparticles or nanoparticles which are then incorporated in the composition. Incorporating the agents in microparticles or nanoparticles can be advantageous for those agents that are reactive with one or more of the components of the composition.

h. Delivery Vehicle

In one aspect of the disclosed implantable composition, the biocompatible graft is a biocompatible polymeric matrix with desired mechanical properties for implantation. However, in other aspects, the biocompatible graft lacks the desired mechanical properties for implantation. Thus, the herein disclosed implantable can be admixed with a delivery vehicle such as a biocompatible polymeric matrix to modify the mechanical properties of the implantable composition. For example, the biocompatible polymeric matrix can be injectable, in situ formable, malleable, or curable. The biocompatible polymeric matrix can also be biodegradable. Examples of biocompatible polymeric matrices for use as delivery vehicles of graft materials are known in the art and disclosed herein.

It is understood that each of the delivery vehicles disclosed herein can in some aspects additionally or alternatively be used as a polymeric tissue graft material in the disclosed compositions and methods. Thus, reference herein to a composition as a delivery vehicle or biocompatible polymeric matrix is also reference to that same composition as a polymeric tissue graft material.

In some aspects, the disclosed delivery vehicle such as a biocompatible polymeric matrix can be a semi-interpenetrating polymer network (“semi-IPN”) composition. U.S. Pat. No. 5,837,752 is incorporated by reference for the teaching of a semi-IPN composition for bone repair comprising (1) a linear polymer selected from the group consisting of linear, hydrophobic biodegradable polymers and linear non-biodegradable hydrophilic polymers; and (2) one or more crosslinkable monomers or macromers containing at least one free radical polymerizable group, wherein at least one of the monomers or macromers includes an anhydride linkage and a polymerizable group selected from the group consisting of acrylate or methacrylate.

In some aspects, the disclosed delivery vehicle such as a biocompatible polymeric matrix can comprise polymerizing anhydride prepolymers. U.S. Pat. No. 5,902,599 is incorporated by reference for the teaching of biodegradable polymer networks formed by polymerizing anhydride prepolymers containing crosslinkable groups, such as unsaturated moieties. The anhydride prepolymers can be crosslinked, for example in a photopolymerization reaction by irradiation of the prepolymer with light in the presence of a photosensitive free radical initiator.

U.S. Patent Publications 2006/0148923 A1 and 2006/0052471 A1 are hereby incorporated herein by reference for the teaching of crosslinkable polymeric materials. For example, the delivery vehicle such as a biocompatible polymeric matrix can comprise a crosslinkable anhydride prepolymer comprising monomers and/or oligomers having polymerizable groups, such as radically polymerizable groups, which crosslink to form a polymer network. Suitable polymerizable groups include unsaturated alkenes (i.e., vinyl groups) such as vinyl ethers, allyl groups, unsaturated monocarboxylic acids, unsaturated dicarboxylic acids, and unsaturated tricarboxylic acids. Unsaturated monocarboxylic acids include acrylic acid, methacrylic acid, and crotonic acid. Unsaturated dicarboxylic acids include maleic, fumaric, itaconic, mesaconic or citraconic acid. The polymerizable groups can be acrylates, diacrylates, oligoacrylates, dimethacrylates, oligomethacrylates, and other biologically acceptable polymerizable groups, such as (Meth)acrylates.

In some aspects, the disclosed delivery vehicle such as a biocompatible polymeric matrix can comprise a thermoplastic system. For example, U.S. Pat. No. 5,278,202 is incorporated by reference for the teaching of a thermoplastic system in which a solid, linear-chain, biodegradable polymer is dissolved in a biocompatible solvent to form a liquid, which can then be administered via a syringe and needle. Examples of biodegradable polymers which can be used in the thermoplastic system include polylactides, polyglycolides, polycaprolactones, polyanhydrides, polyamides, polyurethanes, polyesteramides, polyorthoesters, polydioxanones, polyacetals, polyketals, polycarbonates, polyorthocarbonates, polyphosphazenes, polyhydroxybutyrates, polyhydroxyvalerates, polyalkylene oxalates, polyalkylene succinates, poly(malic acid), poly(amino acids), polyvinylpyrrolidone, polyethylene glycol, polyhydroxycellulose, chitin, chitosan, and copolymers, terpolymers, or combinations or mixtures of the above materials. The polymers can have a lower degree of crystallization and are more hydrophobic. These polymers and copolymers are more soluble in the biocompatible solvents than the highly crystalline polymers such as polyglycolide and chitin which also have a high degree of hydrogen-bonding. Examples of materials with the desired solubility parameters are the polylactides, polycaprolactones, and copolymers of these with glycolide in which there are more amorphous regions to enhance solubility.

In some aspects, the disclosed delivery vehicle such as a biocompatible polymeric matrix can comprise a ceramic composite material. For example, U.S. Pat. No. 6,027,742 is incorporated by reference for the teaching of a bioactive ceramic composite material that is biocompatible, bioresorbable and possesses high strength and/or other desirable mechanical properties. The disclosed bioactive ceramic composite material can be formed at low temperatures, is readily formable and/or injectable, and yet can harden to high strength upon further reaction. The disclosed bioactive ceramic composite material can contain a nano-size, poorly crystalline apatitic calcium phosphate solids with Ca/P ratios comparable to naturally occurring bone minerals and having stiffness and fracture toughness similar to natural bone. The disclosed bioactive ceramic composite material is strongly bioresorbable and its mechanical properties can be adjusted to meet the demands of the particular therapy and/or implant site. The disclosed composite material can be obtained by providing an amorphous calcium phosphate in the presence of a limited quantity of water to produce a hydrated precursor in the form of a putty or paste and promoting the conversion of the amorphous calcium phosphate to a poorly crystalline apatitic calcium phosphate. The conversion is associated with hardening of the paste and can produce a poorly crystalline apatitic calcium phosphate.

The delivery vehicle such as a biocompatible polymeric matrix can comprises cross-linked sodium alginate. Sodium alginate is biocompatibile and in vivo biodegradabe. A sterile and low endotoxin form of sodium alginate is available under product number K8P569 from Monsanto, 800 N. Lindbargh Blvd. St. Louis, Mo., or under product number UP MVG from Pro Nova, Strandveien 18, N-1324 Lysaker, Norway. Very low endotoxin levels can be obtained in alginates by use of a highly specialized purification process. Alginates in a water gel form have the unique ability to form elastic films by reaction with calcium salts and/or magnesium salts. Once cross-linked, these films retain their shape and resist stress.

The delivery vehicle such as a biocompatible polymeric matrix can comprises a UV photo-active polymerhydrogel. Monomers that are polymerizable upon exposure to light radiation have the potential advantage of being formed in vivo at the tissue site of interest via minimally invasive procedures, and can be used as scaffolds in tissue engineering, for cell encapsulation, as drug delivery systems, and as fillers for a tissue defect. See Muggli D S, Burkoth A K, Keyser S A, Lee H R, Anseth K S, “Reaction behavior of biodegradable, photo-cross-linkable polyanhydrides,” Macromolecules 3, 4120-4125 (1998); Lu S, Anseth K S, “Photopolymerization of multilaminated poly(HEMA) hydrogel for controlled release,” J Controlled Release 57, 291-300 (1999); Elisseeff J, Anseth K, Sims D, McIntosh W, Randolph M, Langer R, “Transdermal photopolymerization for minimally invasive implantation,” Proc Natl Acad Sci USA 96(6), 3104-3107 (1999); Burkoth A K, Anseth K S, “A review of photo-crosslinked polyanhydrides: In situ forming degradable networks,” Biomaterials 21(23), 2395-2404 (2000); Elisseeff J, McIntosh W, Anseth K, Riley S, Ragan P, Langer R, “Photoencapsulation of chondrocytes in poly(ethyleneoxide)-based semi-interpenetrating networks,” J Biomed Mater Res 51(2), 164-171 (2000); Cruise G M, Hegre O D, Lamberti F V, Hager S R, Hill R, Scharp D S, Hubbel J A, “In vitro and in vivo performance of porcine islets encapsulated in interfacially photopolymerized poly(ethylene glycol) diacrylate membranes,” Cell Transplant 8(3), 293-306 (2000); Smeds K A, Grinstaff M W, “Photocrosslinkable polysaccharides for in situ hydrogel formation,” J Biomed Mat Res 54(1), 115-121 (2001); all incorporated herein by reference. For example, the polymeric matrix can comprise a polyphosphazene that can be ionically cross-linked or photocured. The polymeric matrix can be a photocurable PEG, such as poly(ethylene glycol) diacrylate (PEGDA).

The delivery vehicle such as a biocompatible polymeric matrix can comprise a hydrogel. The polymeric matrix can be a biocompatible hydrogel comprising at least one polymer. A “hydrogel,” as used herein, refers to a network of polymer chains that are water-soluble, sometimes found as a colloidal gel in which water is the dispersion medium. Hydrogels can be superabsorbent natural or synthetic polymers. For example, hydrogels can contain over 99% water. Hydrogels can also possess a degree of flexibility very similar to natural tissue, due to their significant water content. However, it is also understood that in one aspect, the disclosed hydrogels can comprise water or water mixed with other miscible liquids, for example, alcohols. Hydrogels can comprise positively charged, negatively charged, and neutral hydrogels that can be saturated or unsaturated. Examples of hydrogels are TETRONICS™ and POLOXAMINES™, which are poly(oxyethylene)-poly(oxypropylene) block copolymers of ethylene diamine; polysaccharides, chitosan, poly(vinyl amines), poly(vinyl pyridine), poly(vinyl imidazole), polyethylenimine, poly-L-lysine, growth factor binding or cell adhesion molecule binding derivatives, derivatised versions of the above (e.g. polyanions, polycations, peptides, polysaccharides, lipids, nucleic acids or blends, block-copolymers or combinations of the above or copolymers of the corresponding monomers); agarose, methylcellulose, hydroxyproylmethylcellulose, xyloglucan, acetan, carrageenan, xanthan gum/ocust beangum, gelatine, collagen particularly Type 1), PLURONICS™, POLOXAMERS™, POLY(N-isopropylacrylmide) and N-isopropylacrylmide copolymers. Thus, for example, the at least one polymer can comprise a saccharide residue, an ethylene oxide residue, a propylene oxide residue, an acrylamide residue, or a blend or copolymer thereof. Thus, the at least one polymer can be agarose. The at least one polymer can be a polaxomers, or a derivative thereof. The at least one polymer can be a polyacrylamides, or a derivative thereof. The at least one polymer can be N-isopropylacrylamide (NIPAM), or a derivative thereof. The at least one polymer can be Pluronic F127, or a derivative thereof.

### C. METHODS OF MAKING

A method for preparing a biocompatible implant material, the method comprising contacting a polymeric tissue graft material with an organic solvent and mixing the microsphere with a plurality of nonpolymeric porous particles.

The organic solvent can comprise one or more of dichloromethane, chloroform, carbon tetrachloride, tetrahydrofuran, diethyl ether, ethyl acetate, methanol, ethanol, propanol, isopropanol, butanol, cyclohexanol, cyclohexane, hexane, pentane, heptane, benzene, toluene, or xyxlenes. The organic solvent can comprise a binary solvent. The organic solvent can comprises dichloromethane/ethanol.

The polymeric tissue graft material can be substantially removed from contact with the organic solvent before the mixing step.

The polymeric tissue graft material can comprise a macroporous material. The macroporous material can have pores of greater than about 100 μm. The polymeric tissue graft material can comprises microspheres having diameters of from about 1 μm to about 10,000 μm, from about 50 μm to about 5000 μm, from about 100 μm to about 1000 μm, or from about 50 μm to about 1000 μm. The polymeric tissue graft material can comprise microspheres having diameters of from about 500 μm to about 1000 μm. Thus, the diameters of the microspheres can range from about 500 μm to about 1000 μm.

The polymeric tissue graft material can comprises poly(hydroxyethyl methacrylate) and/or poly(methyl methacrylate). The polymeric tissue graft material can comprise a biodegradable polymer. The polymeric tissue graft material can comprise a nonbiodegradable polymer.

The nonpolymeric porous particles can comprise a microporous material. The microporous material can have pores of less than about 100 μm. The microporous material can have pores of from about 0.5 μm to about 20 μm. The nonpolymeric porous particles can have an average particle size of from about 50 μm to about 500 μm, from about 100 μm to about 400 μm, from about 200 μm to about 300 μm, or from about 180 μm to about 250 μm. The nonpolymeric porous particles can have an average particle size of from about 180 μm to about 250 μm. Thus, the particle sizes of the nonpolymeric porous particles can range from about 180 μm to about 250 μm.

The nonpolymeric porous particles can comprise a nonmetallic inorganic material. The nonpolymeric porous particles can comprise a ceramic. The nonpolymeric porous particles can comprise coral, shell, pearl, or glass. The nonpolymeric porous particles can comprise coral.

The polymeric tissue graft material can comprises a microsphere comprising a poly(hydroxyethyl methacrylate) outer layer and a poly(methyl methacrylate) inner layer and wherein the nonpolymeric porous particles comprise coral particles.

The disclosed method can further comprise the step of sieving the nonpolymeric porous particles before the mixing step. The disclosed method can further comprise the step of drying the implant material after the mixing step. The disclosed method can further comprising the step of sieving the implant material after the mixing step.

Also provided is a method for preparing a biocompatible implant material, the method comprising the steps of contacting a polymeric microsphere with a dichloromethane/ethanol solution and mixing the microsphere with ceramic particles. The polymeric microsphere can comprise poly(hydroxyethyl methacrylate) and/or poly(methyl methacrylate). The ceramic particles can comprise coral.

Also provided is a method for preparing a biocompatible implant material, the method comprising the steps of providing a microsphere comprising an outer layer consisting essentially of poly(hydroxyethyl methacrylate) and an inner layer consisting essentially of poly(methyl methacrylate); roughening the surface of the microsphere by contacting the microsphere with a dichloromethane/ethanol (30%/70% v/v) solution, thereby providing a roughened microsphere; and forming a composite microsphere by exposing the roughened uncoated microsphere to a dichloromethane/ethanol (70%/30% v/v) solution and mixing the roughened uncoated microsphere with coral particles with a particle size of from about 180 μm to about 250 μm.

Also provided is a method for preparing a biocompatible implant material, the method comprising the steps of: providing a microsphere comprising an outer layer consisting essentially of poly(hydroxyethyl methacrylate), an inner layer consisting essentially of poly(methyl methacrylate), and a calcium hydroxide coating; removing the calcium hydroxide coating by contacting the microsphere with an acidic solution for a period of time sufficient to substantially remove the coating, thereby providing a substantially uncoated microsphere; roughening the surface of the microsphere by contacting the microsphere with a dichloromethane/ethanol (30%/70% v/v) solution, thereby providing a roughened uncoated microsphere; and forming a composite microsphere by exposing the roughened uncoated microsphere to a dichloromethane/ethanol (70%/30% v/v) solution and mixing the roughened uncoated microsphere with coral particles with a particle size of from about 180 μm to about 250 μm.

### D. TREATMENT METHODS

The compounds disclosed herein are useful for replace, repair, or reconstruct tissue in the body of a subject. Thus, provided is a method of administering to a subject an implantable composition disclosed herein. Thus, provided is a method of administering to a subject a subject a polymeric tissue graft material having a surface wherein a plurality of nonpolymeric porous particles are disposed at said surface.

The subject of the disclosed methods can be a mammal. The subject can be human. The subject can be a patient. The implantable composition can be administered to a site in the subject in need of replacement, repair, or reconstruction. For example, the composition can be implanted in or adjacent to a bone or joint of the subject. Thus, the subject can have a bone injury.

Also provided is a method comprising administering cells to an implantable composition disclosed herein under conditions suitable to promote cell growth. Thus, provided is a method comprising administering cells to a polymeric tissue graft material having a surface wherein a plurality of nonpolymeric porous particles are disposed at said surface under conditions suitable to promote cell growth. This method can be used in the production of, for example, a prosthesis, such as a prosthetic joint or limb. The cells can be stem cells or progenitor cells. Thus, the cells can be osteoblasts.

Also provided is a method comprising mixing cells with an implantable composition disclosed herein. Thus, provided is a method comprising providing a polymeric tissue graft material having a surface wherein a plurality of nonpolymeric porous particles are disposed at said surface, and mixing cells with the polymeric tissue graft material the under conditions suitable to promote cell growth. The cells can be stem cells or progenitor cells. Thus, the cells can be osteoblasts.

### E. USES

The implantable composition disclosed herein can be used to fill extraction sockets; prevent or repair bone loss due to tooth extraction; repair jaw bone fractures; fill bone voids due to disease and trauma; stabilize an implant placed into an extraction socket and one placed into an edentulous jawbone to provide immediate function (e.g., chewing); provide ridge (of bone) augmentation; repair periodontal bone lesions; and provide esthetic gingiva reshaping and plumping.

The implantable composition disclosed herein can be used to repair bone fractures, fix vertebrae together, repair large bone loss (e.g., due to disease) and provide immediate function and support for load-bearing bones; to aid in esthetics (e.g., chin, cheek, etc.)

The implantable composition disclosed herein can be applied for the above purposes using standard orthopedic or surgical techniques; e.g., it can be applied to a site where bone generation is desired. For example, the implantable composition disclosed herein can be applied into the intervertebral space. The implantable composition disclosed herein can also be pre-cast into a desired shape and size (e.g., rods, pins, screws, plates, and prosthetic devices such as for the skull, chin, and cheek).

The implantable composition disclosed herein can be used to deliver therapeutic or diagnostic agents in vivo. Examples of drugs or agents which can be incorporated into such compositions include proteins, carbohydrates, nucleic acids, and inorganic and organic biologically active molecules. Specific examples include enzymes, antibiotics, antineoplastic agents, local anesthetics, hormones, angiogenic agents, antiangiogenic agents, antibodies, neurotransmitters, psychoactive drugs, drugs affecting reproductive organs, and oligonucleotides such as antisense oligonucleotides.

### F. EXPERIMENTAL

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

The disclosed compositions can enhanced application of tissue graft materials (e.g., Bioplant-HTR) in regenerative medicine through improved cellular interactions. That is, particles (e.g., HTR particles) surface-engineered with nonmetallic inorganic materials (e.g., porous ceramics, coral) exhibit increased porosity, thereby recruiting cells and regenerating tissue (e.g., bone) at a higher rate than unmodified materials.

Bioplant HTR (Hard Tissue Replacement) is a poly(methyl methacrylate) (PMMA) derived hollow microsphere (300, 700 μm diameter).

HTR is made of two layers. The outermost layer is made up of calcium hydroxide, which becomes calcium carbonate apatite when mixed with blood from a surgical site. The inner layer, adjacent to the PMMA surface, is composed of PHEMA (poly(hydroxyethyl)methacrylate).

Blood congeals upon contact with the HTR surface yielding a composite that can be used as a grafting material. Using the grafting abilities of Bioplant-HTR, dentists have been able to place implants into extraction sites to minimize bone atrophy and retain the jawbone's function of holding a root tooth form.

The three-dimensional arrangement of HTR yields a macroporous structure that allows for vacularization and is osteoconductive (i.e., allows new bone formation around the porous particle framework).

1. Cleaning the Surface of the Tissue Graft Material Particles

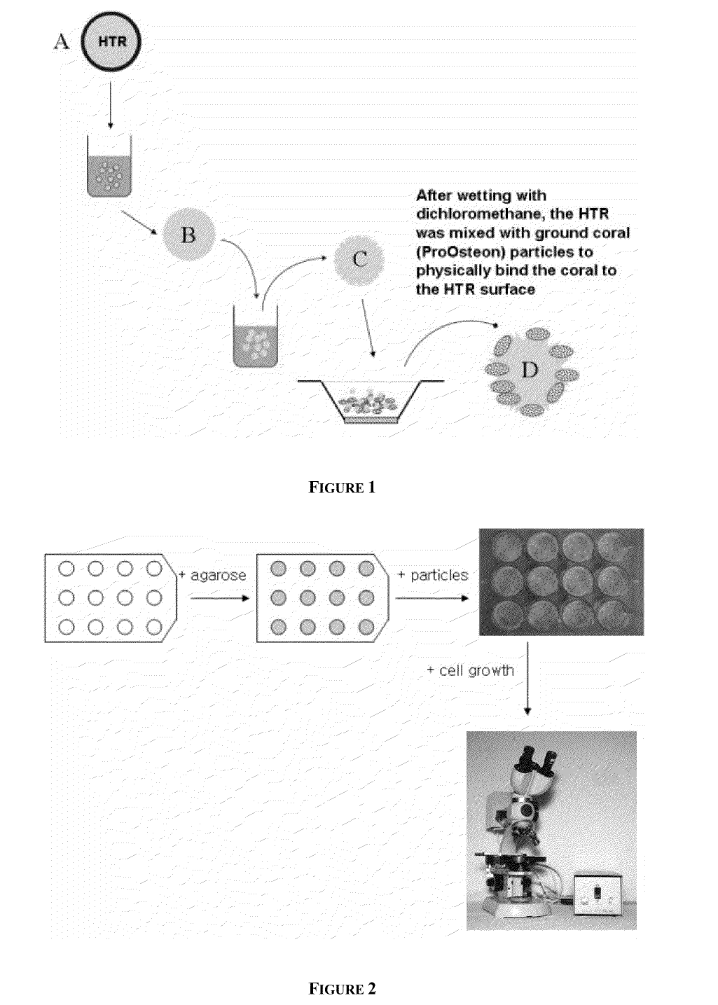

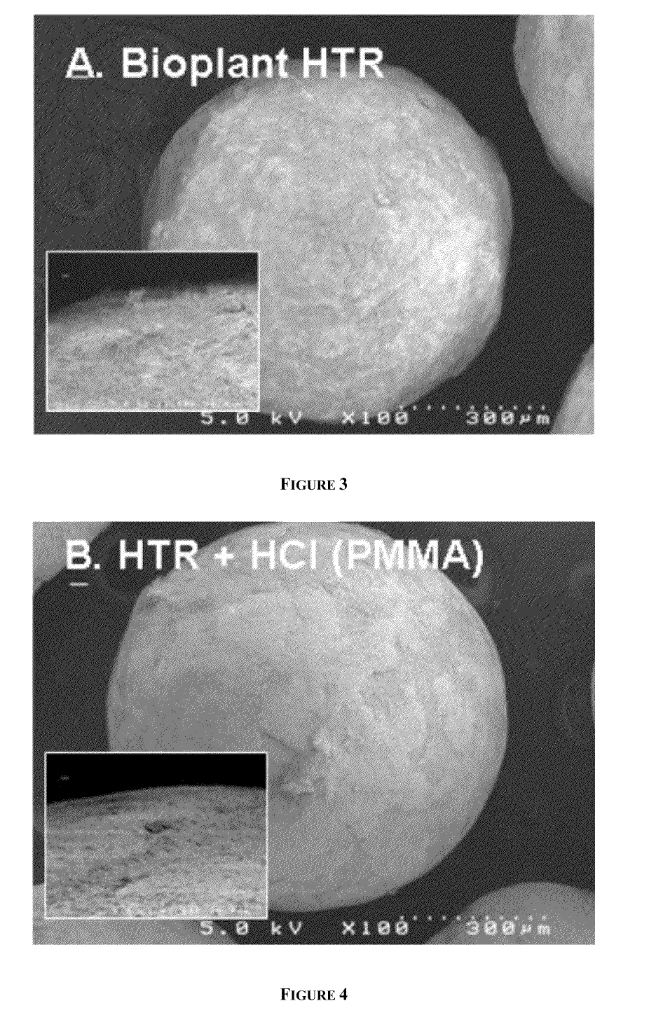

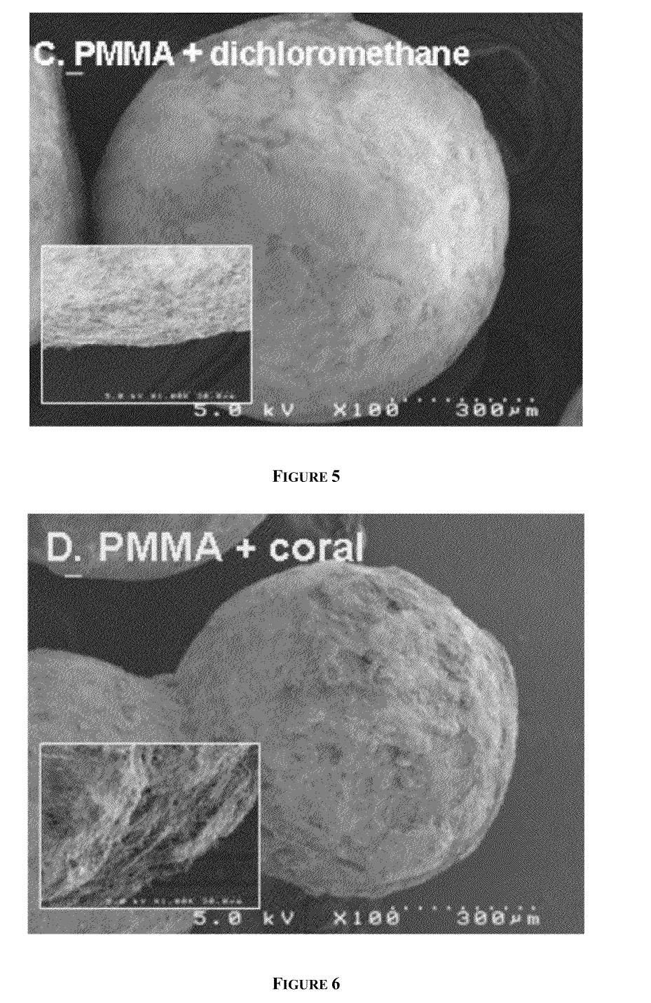

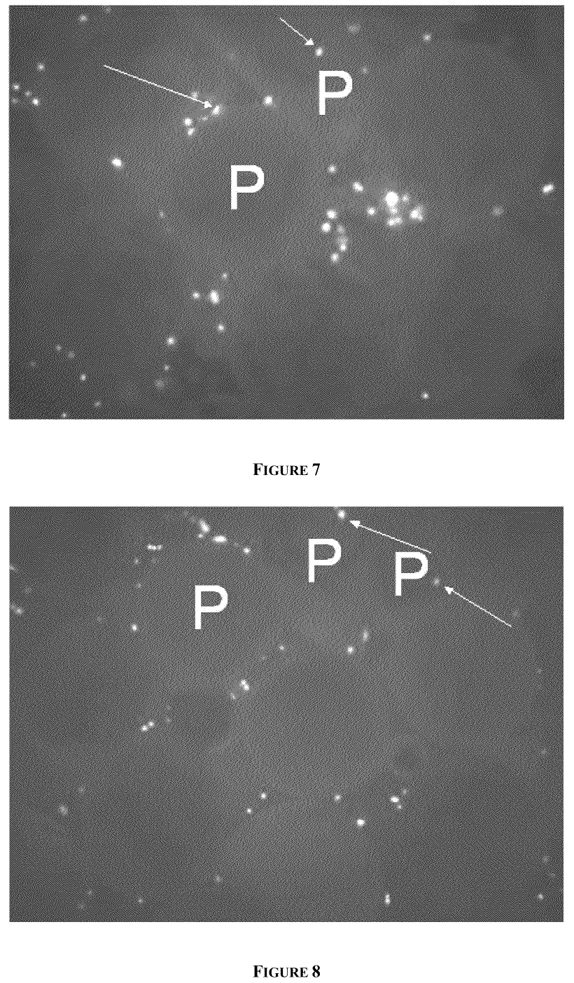

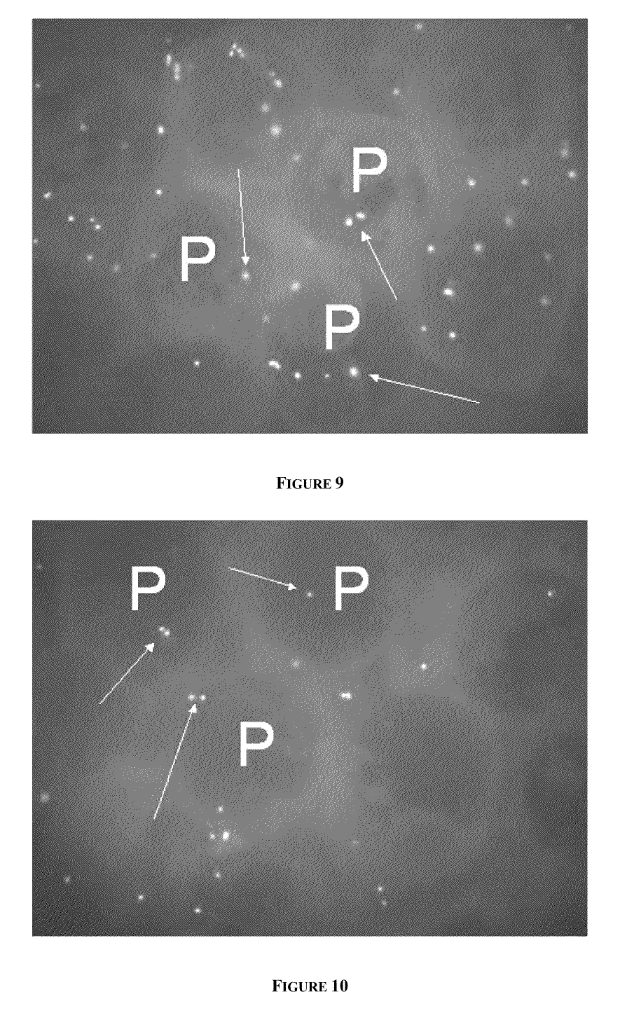

As shown in step A-B of FIG. 1. HTR (5 g) was placed in 100 mL of 0.1N HCl. The particles were allowed to sit overnight in contact with the acidic solution, with constant stirring. The particles were then removed from the solution by filtration and washed three times with deionized water. The particles were then allowed to dry. The process dissolved then outer Ca(OH)₂ layer, leaving the inner polymeric layer.

2. Roughening the Surface of the Uncoated Particles

As shown in step B-C of FIG. 1, the uncoated particles (500 mg) were added to a 100 mL solution of dichloromethane/ethanol (30%/70% v/v) and stirred for one minute, followed by being allowed to sit for ten minutes. The particles were then removed from the solution by filtration and allowed to dry.

3. Making Homogeneous Coral Particles

Coral particles (ProOsteon) were lightly ground in a mortar and pestle. The particles were then passed through a 180 μm sieve, collecting the particles that were not filtered. These particles were then filtered with a 250 μm sieve, collecting the particles that were filtered. This process was repeated until the particles had the desired size range.

4. Making the Composite

As shown in step C-D of FIG. 1, a cell strainer containing a uniform monolayer of coral under a monolayer of polymer particles was immersed into a 10 mL solution of dichloromethane/ethanol (70%/30% v/v) for 15 seconds. The particles were then removed and mixed thoroughly. The mixture was then allowed to dry.

The resulting particles were lightly ground in a mortar and pestle and then passed through a 500 μm sieve, collecting the particles that were not filtered. These particles were then filtered with a 1 mm sieve, collecting the particles that were filtered. This process can repeated until the particles have the desired size range.

5. Procedure for Cell Plates

As shown in FIG. 2, six 12-well plates were coated with 1% agarose (1 mL/well). A monolayer of each type of particle was added to each cell plate row (HTR, polymer (PMMA), PMMA with dichloromethane, PMMA with coral). Three wells of agarose and three empty cells (control variables) were made for all six time periods.

6. Cell Study

Approximately 8000 cells were added to each well (in 1 mL media). The plates were placed in an incubator for pre-determined time points. After taking the cells out of the incubator, the media was collected using a pipette and placed in pre-labeled centrifuge tubes. Another 0.5 mL was added to each tube, and this media was immediately re-collected (washing the particles). 1 mL normal media was added to each well and placed in the incubator once again.

7. Fluorescein Diacetate Imaging Procedure

A fluorescein diacetate stock solution (5 mg/mL) was prepared in acetone. O.1 mL of the stock solution was added to 24.9 mL sterile PBS. 1 mL of the solution was added to the media in each well of the 12-well plate. These were allowed to incubate for 15 minutes. The solution was then aspirated and washed with 1 mL PBS and aspirated again.

Images (2-3 separate fields of view in each well) of both the light and fluorescence views of the same field of vision were taken. The cells were visually counted from each field of view.

The data obtained from the fluorescence images show a trend of increasing cell attachment to the particle surfaces. This trend in complementary to the increasing roughness on the HTR surface after treatment, as observed using SEM. Without wishing to be bound by theory, it is believed than the increased cell attachment can be attributed to improved cell-biomaterial surface interaction, due to increased surface area and nanoscale features.

| | | cells/field | visible cells attached |
|---|---|---|---|
| | FIG. | of vision | to particle surface |
| HTR particles | 7 | 13.9 ± 7 | 46.6% |
| PMMA particles | 8 | 6.75 ± 3.7 | 23.4% |
| PMMA + DCM particles | 9 | 13.9 ± 10 | 69.1% |
| PMMA + coral particles | 10 | 8.1 ± 3 | 70.2% |

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A biocompatible implant material comprising:
a. a polymeric tissue graft material having a surface and
b. a plurality of nonpolymeric porous particles disposed at the surface.

2. The implant material of claim 1, wherein the polymeric tissue graft material comprises a macroporous material.

3. The implant material of claim 1, wherein the polymeric tissue graft material comprises poly(hydroxyethyl methacrylate) and/or poly(methyl methacrylate).

4. The implant material of claim 1, wherein the nonpolymeric porous particles have an average particle size of from about 50 μm to about 500 μm, from about 100 μm to about 400 μm, from about 200 μm to about 300 μm, or from about 180 μm to about 250 μm.

5. The implant material of claim 1, wherein the nonpolymeric porous particles comprise a nonmetallic inorganic material.

6. The implant material of claim 1, wherein the nonpolymeric porous particles comprise coral.

7. The implant material of claim 1, wherein the polymeric tissue graft material comprises a microsphere comprising a poly(hydroxyethyl methacrylate) outer layer and a poly(methyl methacrylate) inner layer and wherein the nonpolymeric porous particles comprise coral particles.

8. A method for preparing a biocompatible implant material, the method comprising the steps of:
a. contacting a polymeric tissue graft material with an organic solvent and
b. mixing the microsphere with a plurality of nonpolymeric porous particles.

9. The method of claim 8, wherein the organic solvent comprises dichloromethane/ethanol.

10. The method of claim 8, wherein the polymeric tissue graft material comprises a macroporous material.

11. The method of claim 8, wherein the polymeric tissue graft material comprises poly(hydroxyethyl methacrylate) and/or poly(methyl methacrylate).

12. The method of claim 8, wherein the nonpolymeric porous particles comprise a nonmetallic inorganic material.

13. The method of claim 8, wherein the nonpolymeric porous particles comprise coral.

14. The method of claim 8, wherein the polymeric tissue graft material comprises a microsphere comprising a poly(hydroxyethyl methacrylate) outer layer and a poly(methyl methacrylate) inner layer and wherein the nonpolymeric porous particles comprise coral particles.

15. A method comprising administering to a subject a polymeric tissue graft material having a surface wherein a plurality of nonpolymeric porous particles are disposed at the surface.

16. The method of claim 15, wherein the polymeric tissue graft material is implanted in or adjacent to a bone or joint of the subject.

17. The method of claim 15, wherein the subject has a bone injury.

18. The method of claim 15, wherein the particles are disposed at said surface under conditions suitable to promote cell growth.

19. The method of claim 18, wherein the cells are stem cells or progenitor cells.

20. The method of claim 19, wherein the cells are osteoblasts.
